(19) United States
(12) Patent Application Publication
(10) Pub. No.: US 2023/0413591 A1
(43) Pub. Date: Dec. 21, 2023

(54) ORGANIC LIGHT-EMITTING DIODE, METHOD FOR PREPARING ORGANIC LIGHT-EMITTING DIODE, DISPLAY PANEL, AND DISPLAY DEVICE

(71) Applicant: BOE Technology Group Co., Ltd., Beijing (CN)

(72) Inventors: Yang LIU, Beijing (CN); Xueqin CHEN, Beijing (CN); Yuqian SUN, Beijing (CN); Lixia QIU, Beijing (CN); Dongxu ZHANG, Beijing (CN)

(21) Appl. No.: 18/036,153

(22) PCT Filed: Nov. 30, 2020
(86) PCT No.: PCT/CN2020/132874

§ 371 Date: May 9, 2023

(51) Int. Cl. H10K 50/12; H10K 50/18; H10K 71/60; H10K 85/60

(52) U.S. Cl. CPC H10K 50/12; H10K 50/181; H10K 71/60; H10K 85/633; H10K 85/622; H10K 85/6574; H10K 2101/40

(57) ABSTRACT

An organic light-emitting diode includes: an anode, a cathode and a light-emitting layer between the anode and the cathode. The light-emitting layer has a blue light host material; on the side of the light-emitting layer facing the anode, an electron blocking layer is provided in the direction away from the light-emitting layer; the electron blocking layer has a hole material; and the hole material has a structural formula as shown in the following formula (I), and the blue light host material has a structural formula as shown in the following formula (II).

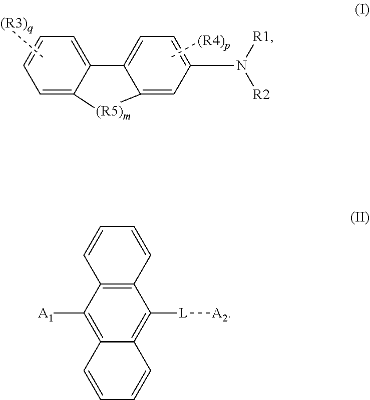

### CROSS-REFERENCE TO RELATED APPLICATION

This application is a national stage application of PCT Application No. PCT/CN2020/132874, which is filed on Nov. 30, 2020, and entitled “Organic Light-Emitting Diode, Method for Preparing Organic Light-Emitting Diode, Display Panel, and Display Device”, the content of which should be regarded as being incorporated herein by reference.

### TECHNICAL FIELD

The present disclosure relates to the field of display technology, and in particular to an organic light-emitting diode, a method for preparing an organic light-emitting diode, a display panel, and a display device.

### BACKGROUND

With the development of display technology, display devices based on organic light-emitting diodes (OLEDs) have been more widely used. In the current organic light-emitting diode devices, phosphorescent devices can be used for red light and green light devices, and specifically, their light-emitting hosts are all double-host materials. However, due to the lag in technological development of blue phosphorescent materials, most of the mass-produced blue light devices are fluorescent devices, and the blue light host material is a single-host material. The host materials (referred to as B host for short) for fluorescent blue light are mostly derivatives of anthracene, which leads to electron materials as the host of blue light. In order to improve performances of OLED devices, there are also structures in the devices, including electron blocking layer, hole injection layer and electron transport layer. The hole injection material (referred to as B prime for short) in the electron blocking layer adjacent to the light-emitting layer is a hole material, which is beneficial to the injection of holes from the electron blocking layer to the light-emitting layer on the one hand, and the blockage of electrons on the other hand, preventing electron transport from the light-emitting layer to the electron blocking layer, limiting the recombination of excitons in the light-emitting layer and ensuring luminous efficiency. However, because the B host being the electron material more easily induces accumulation of electrons at the B host/B prime interface and the B prime being the hole material induces accumulation of holes at the B host/B prime interface, the exciton recombination region is also concentrated at the B host/B prime interface. The exciton recombination at B host/B prime interface will induce some problems, such as accelerating the aging of materials at the interface and affecting the performance and lifetime of light-emitting devices.

Therefore, the current organic light-emitting diodes, methods for preparing organic light-emitting diodes, display panels and display devices still need to be improved.

### SUMMARY

The present disclosure aims at alleviating or solving at least to some extent at least one of the above mentioned problems.

In one aspect of the present disclosure, the present disclosure provides an organic light-emitting diode. The organic light-emitting diode includes: an anode, a cathode and a light-emitting layer between the anode and the cathode, the light-emitting layer having a blue light host material, a side of the light-emitting layer facing the anode having an electron blocking layer in a direction away from the light-emitting layer, the electron blocking layer having a hole material, the hole material having the structural formula as shown in the following formula (I), and the blue light host material having the structural formula as shown in the following formula (II):

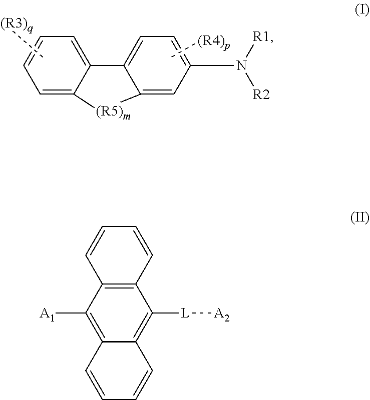

Wherein, q and p are each independently 1, 2 or 3, R1-R4 are each independently selected from H, C6-C50 aryl, C6-C50 aromatic heterocyclic, C6-C50 alkyl, C6-C50 alkoxy, C6-C50 aralkyl, C6-C50 aryloxy, C6-C50 arylthio, C6-C50 alkoxy, carbonyl, carboxyl, halogen, cyano, nitro, and hydroxyl, and at least one of R1 and R2 is a large steric hindrance group containing not less than 12 carbon atoms, and R3 and R4 are not simultaneously H; m is 0 or 1, and R5 is phenyl or biphenyl; A1 and A2 are each independently selected from substituted or unsubstituted C6-C50 Aryl, and L is a single bond, phenylene or naphthene. The organic light-emitting diode can alleviate or even prevent the formation of exciplex through the selection of blue light host material and hole material, thus alleviating or even preventing problems caused by the recombination of excitons at the B host/B prime interface.

According to an embodiment of the present disclosure, R1 to R4 are each independently selected from the group consisting of H, C6-C20 aryl, C6-C20 aromatic heterocyclic, C6-C20 alkyl, C6-C20 alkoxy, C6-C20 aralkyl, C6-C20 aryloxy, C6-C20 arylthio group, C6-C20 alkoxy, carbonyl, carboxyl, halogen atom, cyano, nitro and hydroxyl. Therefore, the formation of exciplex can be better prevented.

According to an embodiment of the present disclosure, the large steric hindrance group includes at least one selected from the group consisting of biphenyl, triphenyl, o-terphenyl, m-terphenyl, p-terphenyl, spirofluorene, fused dibenzofuran, fused dibenzothiophene, spiroxanthene, adamantane, and soccer olefin. Therefore, the performance of the organic light-emitting diode can be further improved.

According to an embodiment of the present disclosure, both R1 and R2 are the large steric hindrance groups. Thereby, the intermolecular distance between the hole material and the blue light host material can be further increased.

According to an embodiment of the present disclosure, m is 1 and the sum of q and p is 2. Therefore, the performance of the organic light-emitting diode can be further improved.

According to an embodiment of the present disclosure, one of R3 and R4 is H, and the other is one selected from the group consisting of spirofluorene, fused dibenzofuran, fused dibenzothiophene and spiroxanthene.

According to an embodiment of the present disclosure, both R1 and R2 are biphenyl groups. Therefore, the performance of the organic light-emitting diode can be further improved.

According to an embodiment of the present disclosure, L is a single bond, and A1 and A2 are each independently fused aromatic ring groups with C number not less than 18. Therefore, the performance of the organic light-emitting diode can be further improved.

According to an embodiment of the present disclosure, the difference between HOMO of the hole material and HOMO of the blue light host material, and the difference between LUMO of the hole material and LUMO of the blue light host material satisfy: ΔHOMO≤0.3 eV, ΔLUMO≥0.4 eV. Therefore, the exciton recombination region can be alleviated or even prevented from being concentrated at the B host/B prime interface.

According to an embodiment of the present disclosure, a intermolecular distance between a HOMO unit of the hole material and a LUMO unit of the blue light host material is more than or equal to 4 amies. Therefore, the generation of intermolecular charge transfer excitons (CT state excitons) can be alleviated or even prevented.

According to an embodiment of the present disclosure, a ratio of the hole mobility of the hole material to the electron mobility of the blue light host material is not less than 10.

Therefore, the exciton recombination region can be alleviated or even prevented from being concentrated at the B host/B prime interface.

According to an embodiment of the present disclosure, in a direction from the anode to the cathode, the organic light-emitting diode includes:
a hole injection layer, an electron blocking layer, the light-emitting layer, a hole blocking layer, an electron transport layer and an electron injection layer, wherein the light-emitting layer includes the blue light host material and a doped material, and the doping ratio of the doped material is 1% to 5%. Therefore, the performance of the organic light-emitting diode can be further improved.

In another aspect of the present disclosure, a method for preparing the organic light-emitting diode described above is provided. The method includes the following steps: forming an anode on a substrate; forming an electron blocking layer on a side of the anode away from the substrate, and forming a light-emitting layer on a side of the electron blocking layer away from the anode; forming a cathode on a side of the light-emitting layer away from the anode. The method can easily obtain the organic light-emitting diode.

In another aspect of the present disclosure, the present disclosure provides a display panel. The display panel includes a substrate having multiple organic light-emitting diodes on the substrate, a part of the multiple organic light-emitting diodes being those described above. Thus the display panel has all the features and advantages of the organic light-emitting diodes described above and will not be repeated here.

In another aspect of the present disclosure, the present disclosure provides a display device. The display device includes the display panel described above. Thus the display device has all the features and advantages of the organic light-emitting display panel described above and will not be repeated here.

### BRIEF DESCRIPTION OF DRAWINGS

The above mentioned and/or additional aspects and advantages of the present disclosure will become apparent and easy to understand from following description of embodiments in conjunction with accompanying drawings.

FIG. 1 shows a schematic diagram of a structure of an organic light-emitting diode according to an embodiment of the present disclosure;

FIG. 2 shows a schematic diagram of a structure of an organic light-emitting diode according to another embodiment of the present disclosure;

FIG. 3 shows a schematic diagram of the potential energy surface of the exciplex and emission in the related art;

FIG. 4 shows a diagram of spectrum test results of Example 1;

FIG. 5 shows the radial distribution function of Example 1;

FIG. 6 shows a diagram of spectrum test results of Comparative Example 1; and

FIG. 7 shows the radial distribution function of Comparative Example 1.

### DETAILED DESCRIPTION

Embodiments of the present disclosure will be described in detail below, examples of the embodiments are shown in the accompanying drawings, wherein the same or similar reference numerals refer to the same or similar elements or elements with the same or similar functions throughout. Embodiments described herein with reference to the accompanying drawings are exemplary, used for explaining the present disclosure only, but should not be construed to limit the present disclosure.

In one aspect of the present disclosure, the present disclosure provides an organic light-emitting diode. Referring to FIG. 1, the organic light-emitting diode includes: an anode 300, a cathode 100, and a light-emitting layer 200 between the anode and the cathode, the light-emitting layer 200 having a blue light host material, a side of the light-emitting layer 200 facing the anode 300 having an electron blocking layer 400, the electron blocking layer 400 having a hole material with a structural formula as shown in the following formula (I), the blue light host material having a structural formula as shown in the following formula (II):

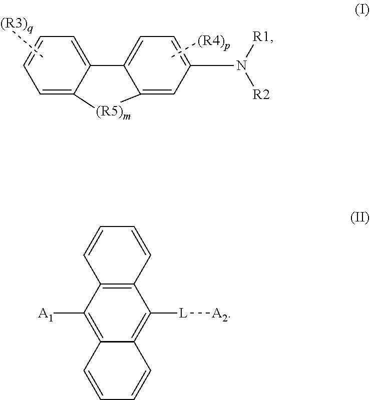

wherein q and p are each independently 1, 2 or 3, R1-R4 are each independently selected from H, C6-C50 aryl, C6-C50 aromatic heterocyclic, C6-C50 alkyl, C6-C50 alkoxy, C6-C50 aralkyl, C6-C50 aryloxy, C6-C50 arylthio, C6-C50 alkoxy, carbonyl, carboxyl, halogen, cyano, nitro, and hydroxyl, and at least one of R1 and R2 is a large steric hindrance group containing not less than 12 carbon atoms, and R3 and R4 are not simultaneously H; m is 0 or 1, and R5 is phenyl or biphenyl; A1 and A2 are each independently selected from substituted or unsubstituted C6-C50 Aryl, and L is a single bond, phenylene or naphthene. The organic light-emitting diode can alleviate or even prevent the formation of exciplex through the selection of blue light host material and hole material, thus alleviating or even preventing the problem caused by the recombination of excitons at the B host/B prime interface.

For convenience of understanding, the principle that the organic light-emitting diode can achieve the aforementioned beneficial effects is briefly explained below:

As mentioned earlier, because fluorescent blue light host materials in blue light organic light-emitting diodes is a multi-electron material and the materials in the electron blocking layer are mostly hole materials, electrons and holes are easy to gather at the B host/B prime interface. However, when blue light host materials and hole materials meet certain requirements, holes at the HOMO energy level in the hole materials and electrons at LUMO energy level in the blue light host materials are easy to form intermolecular charge transfer excitons (CT state excitons) and exciplex. In addition to accelerated aging of materials at the interface, exciton emission wavelengths are significantly red-shifted compared to B prime and B host. The energy transfer efficiency is determined by the overlapping area between the emission spectrum of the B host and the absorption spectrum of the doped material in the light-emitting layer. Therefore, if the exciplex is formed, the overlapping area between the red-shift emission spectrum and the energy spectrum of doped materials will be weakened to a certain extent, which leads to the decrease in the energy transfer efficiency, and then leads to the decrease in luminous efficiency of devices. The inventor found that whether exciplex is formed or not has a great relationship with the intermolecular distance between B host and B prime. Specifically, referring to FIG. 3, with reference to the schematic diagram of the potential energy surface of the exciplex and the emission, it can be seen that the energy (A+D) of the ground state of the electron withdrawing group and the electron donating group forming the exciplex and the energy (A+D*) of the excited state of the electron withdrawing group and the electron donating group are dependent on the atomic spacing (R) between the electron withdrawing group and the electron donating groupAD). When the distance between the two atoms is less than a certain distance, the energy band (A+D*) of the excited states of the electron-withdrawing group and the electron-donating group is bent, which is easy to produce the above-mentioned intermolecular charge transfer excitons. Compared with the emission spectrum of normal blue light emission process (as shown in Figure hvD), the spectrum of exciplex luminescence shifts to the left, that is, a red shift occurs. Usually, the doped materials in the light-emitting layer are matched by the position of emission spectrum. Obviously, the overlap between exciplex spectrum and absorption spectrum of doped materials decreases after red shift, and the formation of exciplex also affects the luminescent efficiency of the organic light-emitting diode. Generally speaking, the inventors found that when the distance between the two molecules is greater than 4 amies, it is difficult to form exciplex.

According to embodiments of the present disclosure, R1-R4 each may be independently selected from H, C6-C20 aryl, C6-C20 aromatic heterocyclic, C6-C20 alkyl, C6-C20 alkoxy, C6-C20 aralkyl, C6-C20 aryloxy, C6-C20 arylthio, C6-C20 alkoxy, carbonyl, carboxyl, halogen, cyano, nitro, and hydroxyl in order to reasonably control the intermolecular distance between the photo-host material and the hole material. Therefore, the formation of exciplex may be better prevented.

In an embodiment of the present disclosure, the alkyl may be saturated or unsaturated alkyl (e.g. alkenyl and alkynyl), linear or branched alkyl, linear or branched saturated alkyl, and linear or branched unsaturated alkyl. The alkyl may also have one or more halogenated substitutions i.e. the alkyl in the present disclosure may include halogenated alkyl. Specifically, it may be at least one of saturated alkyl, alkenyl, alkynyl and haloalkyl. The term “haloalkyl” means that an alkyl is substituted by one or more halogen atoms, examples of which include, but are not limited to, chloroalkyl, bromoalkyl or fluoroalkyl.

The term “aryl” represents monocyclic, bicyclic and polycyclic carbocyclic systems containing 6 or more ring atoms, wherein at least one ring system is aromatic. Examples of aryl groups may include phenyl, naphthyl, biphenyl, and fused aromatic ring groups. The term “aromatic heterocyclic group” represents containing at least one aromatic heterocyclic ring, and the heteroatom can be oxygen, sulfur and so on. The aromatic heterocyclic group has at least one ring-closed conjugated system. The molecules in the conjugated system are planar, and there are ring-shaped delocalized electron clouds on the upper and lower sides of this plane, and the number of P electrons in the conjugated system follows Hock rule. The term “aralkyl” represents alkyls containing one or more aromatic substituents in an alkyl chain, e.g. H atoms in a linear or branched carbon chain are substituted by the aryls including, but not limited to, the aforementioned. The term “aryloxy” represents an oxygen atom group to which an aromatic ring structure is attached, and specifically, one side of the oxygen atom may be attached to the benzene ring shown in structural formula (I), and the other side may be attached to the aromatic ring structure. Similarly, the term “arylthio” represents an S atom group to which an aromatic ring structure is attached, and in particular, the benzene ring shown in structural formula (I) may be attached to one side of the S atom and the aromatic ring structure may be attached to the other side.

The term “alkoxy” represents groups consisting of an oxygen atom and an alkyl group, which may have the meaning of the aforementioned term “alkyl” and be linked to the structural formula as shown in formula (I) through an oxygen atom.

According to an embodiment of the present disclosure, in order to further improve the steric hindrance between the blue light host material and the hole material and ensure the intermolecular distance between them, at least one of R1 and R2 can be made to be a large steric hindrance group. Specifically the large steric hindrance group may include a substituent consisting of not less than 12 carbon atoms and having a cyclic structure. In particular, at least one selected from the group consisting of biphenyl, triphenyl, o-terphenyl, m-terphenyl, p-terphenyl, spirofluorene, fused dibenzofuran, fused dibenzothiophene, spiroxanthene, adamantane, and soccer olefins may be included. Therefore, the performance of the organic light-emitting diode can be further improved.

According to some specific embodiments of the present disclosure, R1 and R2 may both be the large steric hindrance group. Therefore, the intermolecular distance between the hole material and the blue light host material may be further increased.

According to some embodiments of the present disclosure, m may be 0 or 1. That is, the hole material in the present disclosure may not contain an R5 group or may contain an R5 group. R5 may be phenyl or biphenyl. That is, the two benzene rings shown in formula (I) may also form chemical bonds with the phenyl or biphenyl groups of R5 in the manner shown in formula (I). Therefore, the steric hindrance between the hole material and the blue light host material can be further improved, and the intermolecular distance can be increased.

According to some specific embodiments of the present disclosure, in order to reduce the difficulty and cost of synthesis, one of R3 and R4 may be H and the other one selected from the group consisting of spirofluorene, fused dibenzofuran, fused dibenzothiophene and spiroxanthene, provided that there is at least one large steric hindrance group in R1 and R2. The replacement position of R3 or R4 is not particularly limited and can be selected by a person skilled in the art according to the actual situation. In some embodiments, R3 may be located in a position aligned with the N atom. R4 can be located on the side of benzene ring far away from R5, that is, it can be located at the meta-position or para-position of R5.

According to some specific embodiments of the present disclosure, m may be 1 and the sum of q and p is 2. Specifically, R5 may be a biphenyl group. Therefore, the performance of the organic light-emitting diode can be further improved. According to embodiments of the present disclosure, R1 and R2 may both be biphenyl groups. Therefore, the performance of the organic light-emitting diode can be further improved.

The inventors found that the hole material with the above structure has good hole transport and electron blocking properties on the one hand, and can maintain sufficient intermolecular distance with the blue light host material on the other hand to prevent the generation of the exciplex mentioned above.

According to the embodiment of the present disclosure, the blue light host material described in the present disclosure is not particularly limited, and the technical personnel in the field can select according to the device performance requirements and the specific structure of the hole material, for example, the structure formula as shown in the aforementioned formula (II) can be provided. In particular, L in the structural formula shown in formula (II) may be a single bond, and A1 and A2 are each independently C6-C50 aryl. More specifically, it may be a fused aromatic ring group having a C number of not less than 18. Therefore, the performance of the organic light-emitting diode can be further improved. For example, according to some specific embodiments of the present disclosure, A1 and A2 may be each independently phenyl, naphthyl, triphenyl, fluoranthene, fused cyclic carbazole, fused dibenzofuran, and dibenzothiophene.

According to the embodiment of the present disclosure, on the premise that the hole material and the blue light host material satisfy the aforementioned structure, the difference between HOMO of the hole material and HOMO of the blue light host material, and the difference between LUMO of the hole material and LUMO of the blue light host material can satisfy:

ΔHOMO≤0.3 eV,ΔLUMO≥0.4 eV.

Thus, the formation of an exciplex at the interface of the electron blocking layer and the light-emitting layer can be alleviated or even prevented on the premise that the energy level matching of the electron blocking layer and the light-emitting layer is ensured.

According to an embodiment of the present disclosure, the intermolecular distance between the HOMO unit of the hole material and the LUMO unit of the blue light host material can be controlled to be greater than or equal to 4 amies. Therefore, the generation of intermolecular charge transfer excitons (CT state excitons) can be alleviated or even prevented.

According to an embodiment of the present disclosure, the ratio of the hole mobility of the hole material to the electron mobility of the blue light host material is not less than 10.

Therefore, the exciton recombination region can be alleviated or even prevented from being concentrated at the B host/B prime interface.

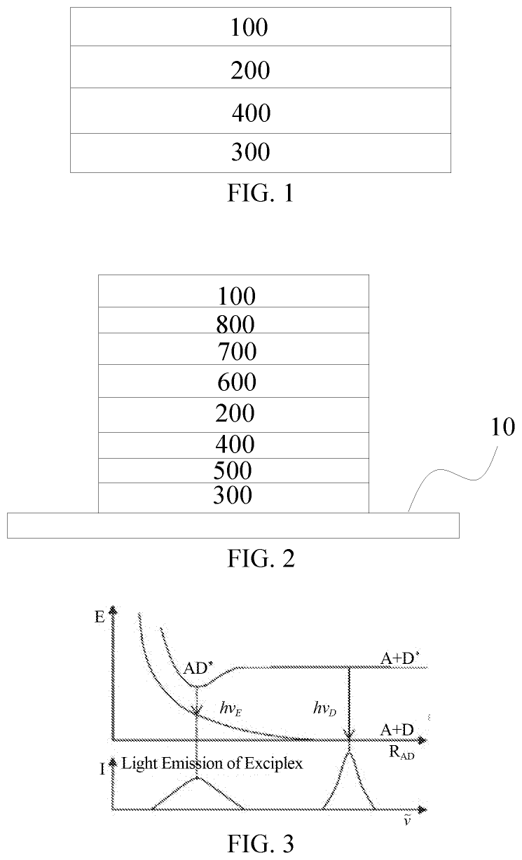

According to an embodiment of the present disclosure, referring to FIG. 2, in order to further improve the performance of the organic light-emitting diode, the organic light-emitting diode may further have a structure such as a hole injection layer 500, in particular, the hole injection layer 500 may be located between the anode 300 and the light-emitting layer 200, and in particular may be located on the side of the anode 300 away from the substrate 10. The electron blocking layer 400 may be located on a side of the hole injection layer 500 away from the anode 300 and the light-emitting layer 300 may be adjacent to the electron blocking layer 400. The hole blocking layer 600 may be located on the side of the light-emitting layer 300 away from the anode 300 and the electron transport layer 700 may be located on the side of the hole blocking layer 600 away from the light-emitting layer 300. The electron injection layer 800 is located on the side of the Electron Transport Layer 700 away from the hole blocking layer 600 and the cathode 100 may be located on the side of the electron injection layer 800 away from the electron transport layer 700. Therefore, the performance of the organic light-emitting diode can be further improved.

In another aspect of the present disclosure, a method of preparing the organic light-emitting diodes described above is provided. The method includes the operations of forming an anode on a substrate, forming an electron blocking layer on a side of the anode remote from the substrate, forming a light-emitting layer on a side of the anode, and forming a cathode on a side of the light-emitting layer remote from the anode. The method can easily obtain the organic light-emitting diode.

According to the embodiment of the present disclosure, the specific operation of forming the anode, the light-emitting layer, the cathode, and the electron blocking layer is not particularly limited, and a person skilled in the art can select a method including but not limited to sputtering deposition, vacuum evaporation, etc. to form the aforementioned structure according to actual needs. According to other embodiments of the present disclosure, the method may also include the operation of forming a structure such as a hole blocking layer, an electron transport layer, an electron blocking layer, etc. The specific positions of the structure such as the hole blocking layer, the electron transport layer, the electron blocking layer, etc. have been described in detail above and will not be repeated herein.

In another aspect of the present disclosure, the present disclosure provides a display panel. The display panel includes a substrate having multiple organic light-emitting diodes on the substrate, a part of the multiple organic light-emitting diodes being those described above. Thus the display panel has all the features and advantages of the organic light-emitting diodes described above and will not be repeated here.

In another aspect of the present disclosure, the present disclosure provides a display device. The display device includes the display panel described above. Thus the display device has all the features and advantages of the organic light-emitting display panel described above and will not be repeated here.

The present disclosure is described below by specific examples which will be understood by those skilled in the art for purposes of illustration only and not in any way limiting the scope of the application. In addition in the following examples the materials and equipment employed are commercially available unless otherwise specified. If specific processing conditions and processing methods are not explicitly described in later examples processing may be performed using conditions and methods well known in the art.

### Example 1

The structural formulas of the hole molecule P1, the electron blue light host material N1 and the doped material BD are as follows:

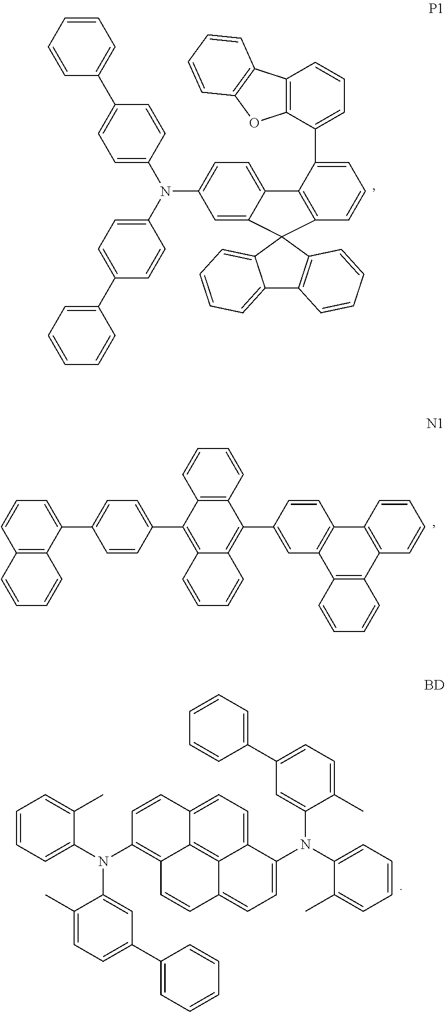

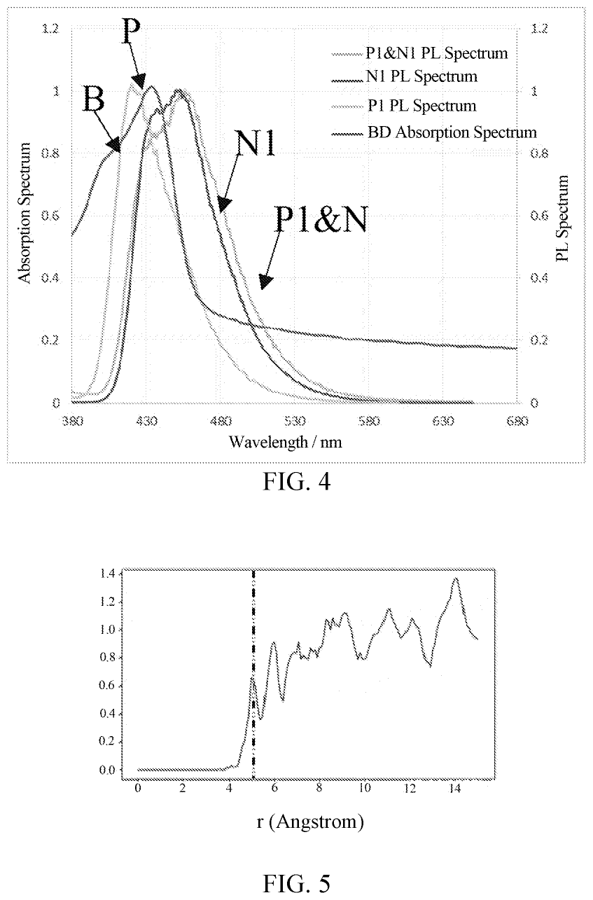

A 50 nm doped film was deposited by vapor deposition of P1 and N1 molecules at a molar ratio of 1:1. The spectral measurements were compared with those of 50 nm films of P1 molecules and 50 nm films of N1 molecules. Referring to FIG. 4, the emission spectra of the P1-N1 doped film have no significant red shift compared with those of the N1 film. Moreover, the spectra of P1-N1 doped films have a good overlapping area at the absorption peak of BD film, and the spectra have an overlapping area of 72%, which proves that the energy transfer efficiency from P1-N1 doped films to BD is very high.

The carrier mobility, HOMO and LUMO levels of P1 and N1 films with the above structure were measured. The results are as follows:

HOMOₚ₁−HOMO_{N1}=0.2 eV,

LUMOₚ₁−LUMO_{N1}=0.5 eV;

Hole Mobility μ of P_1_{H}=5.6×10^{0.5}; Electron mobility μ of N1_{E}=7.3×10⁻⁶.

Referring to FIG. 5, from the radial distribution function of P1 and N1, it can be seen that the intermolecular distance between them is about 5 amies.

### Comparative Example 1

The structural formulas of hole molecule P2, electron blue light host material N2 and doped material BD are as follows:

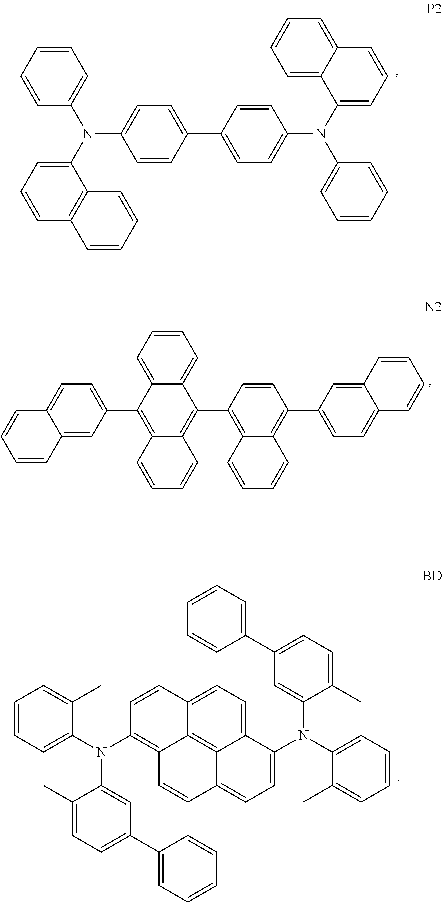

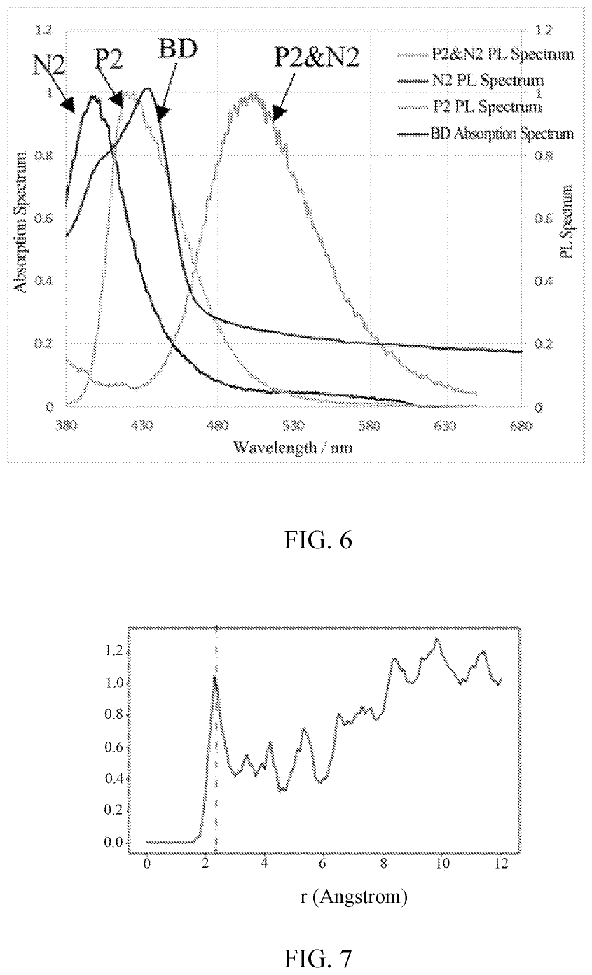

A 50 nm doped film was prepared by evaporation of P2 and N2 molecules at a molar ratio of 1:1. The spectral measurements were compared with those of 50 nm film of P1 molecule and 50 nm film of N1 molecule. Referring to FIG. 6, the emission spectrum of P2-N2 doped film is obviously red-shifted compared with that of N2 film, and the overlapping area of the emission spectrum of P2-N2 doped film at the absorption peak of BD film is greatly reduced compared with that of N2 film, indicating that the energy transfer efficiency from P2-N2 doped film to BD is very low, and exciplex recombination occurs.

The carrier mobility, HOMO and LUMO levels of P2 and N2 films with the above structure were measured. The results are as follows:

|HOMO_{P1}−HOMO_{N1}|=0.4 eV,

|LUMO_{P1}−LUMO_{N1}|=0.3 eV;
The hole mobility μₕ of P1=4.2×10⁻⁵; electron mobility μₑ of N1=6.1×10⁻⁴.

Referring to FIG. 7, it can be seen from the radial distribution function of P1 and N1 that the intermolecular distance between them is about 2.3 amies.

### Example 2

The anode is ITO on which a hole injection layer (HTL), an electron blocking layer (EBL), a light-emitting layer (EML), a hole blocking layer (HBL), an electron transport layer (ETL), an electron injection layer (EIL) and a cathode are formed in turn.

The EBL contains hole materials represented by P1, the EML contains an electron blue light host material N1, and 3% of the doped material BD, and the thickness of the EML is 20 nm.

### Comparative Example 2

The remaining structure is the same as that of Example 2, except that the EBL contains the hole material represented by P2, and the EML contains the blue light host material N2 of the electron, and 3% of the doped material BD.

The organic light-emitting diodes prepared in Example 2 and Comparative Example 2 were subjected to IVL and lifetime tests, voltage, lumen efficiency (Cd/A), chrominance efficiency (Cd/A/CIE y), color coordinates (CIE x and CIE y), and lifetime (LT95) of luminance attenuation to 95%. Based on the results measured in Example 2, the test results of Example 2 and Comparative Example 2 are as follows in Table 1:

TABLE 1

| | | | Cd/A/ | CIE | CIE | |
|---|---|---|---|---|---|---|
| | V | Cd/A | CIE y | x | y | LT95. |
| Example 2 | 100% | 100% | 100% | 0.135 | 0.131 | 100% |
| Comparative | 105% | 62% | 61% | 0.135 | 0.133 | 72% |
| Example 2 | | | | | | |

By comparison, it can be seen that LT95, Cd/A/CIE y and efficiency of Example 2 are significantly higher than those of Comparative Example 2 without significant shift of color coordinates, and the lighting voltage of devices is lower than that of Comparative Example 2. Specifically, the lumen efficiency of Comparative Example 2 was only 62% of that of Example 2, and the LT95 lifetime was only 72% of that of Example 2. Therefore, the light-emitting diode of Example 2 has better stability and better lifetime.

In the description of this specification, description referring to terms “one embodiment”, “another embodiment”, etc. means that specific features, structures, materials, or characteristics described in connection with this embodiment are contained in at least one embodiment of the present application. In this specification, the schematic expression of the above terms does not necessarily refer to the same embodiment or example. Moreover, the specific feature, structure, material, or characteristic described may be combined in a proper way in any one or more embodiments or examples. In addition, without a conflict, a person skilled in the art may combine different embodiments or examples described in this specification and the features of different embodiments or examples.

Although the embodiments of the present application have been shown and described above, it should be understood that the above embodiments are exemplary, but will not be understood as the limitation to the present application. Alterations, modifications, substitutions and variations to the above embodiments may be made by those skilled in the art within the scope of the present application.

1. An organic light-emitting diode comprising: an anode, a cathode and a light-emitting layer between the anode and the cathode,
the light-emitting layer having a blue light host material,
a side of the light-emitting layer facing the anode having an electron blocking layer in a direction away from the light-emitting layer,
the electron blocking layer having a hole material,
the hole material having the structural formula as shown in the following formula (I), and
the blue light host material having the structural formula as shown in the following formula (II): 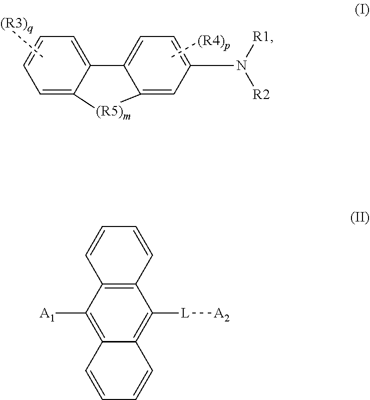
wherein, q and p are each independently 1, 2 or 3, R1-R4 are each independently selected from H, C6-C50 aryl, C6-C50 aromatic heterocyclic, C6-C50 alkyl, C6-C50 alkoxy, C6-C50 aralkyl, C6-C50 aryloxy, C6-C50 arylthio, carbonyl, carboxyl, halogen, cyano, nitro, and hydroxyl, and at least one of R1 and R2 is a large steric hindrance group containing not less than 12 carbon atoms, and R3 and R4 are not simultaneously H;
m is 0 or 1, and R5 is phenyl or biphenyl;
A1 and A2 are each independently selected from substituted or unsubstituted C6-C50 aryl, and L is a single bond, phenylene or naphthene.

2. The organic light-emitting diode according to claim 1, wherein the difference between HOMO of the hole material and HOMO of the blue light host material, and the difference between LUMO of the hole material and LUMO of the blue light host material satisfy:
ΔHOMO≤0.3 eV,
ΔLUMO≥0.4 eV.

3. The organic light-emitting diode according to claim 1, wherein a intermolecular distance between a HOMO unit of the hole material and a LUMO unit of the blue light host material is more than or equal to 4 amies.

4. The organic light-emitting diode according to claim 1, wherein a ratio of the hole mobility of the hole material to the electron mobility of the blue light host material is not less than 10.

5. The organic light-emitting diode according to claim 1, wherein R1 to R4 are each independently selected from the group consisting of H, C6-C20 aryl, C6-C20 aromatic heterocyclic, C6-C20 alkyl, C6-C20 alkoxy, C6-C20 aralkyl, C6-C20 aryloxy, C6-C20 arylthio group, C6-C20 alkoxy, carbonyl, carboxyl, halogen atom, cyano, nitro and hydroxyl.

6. The organic light-emitting diode of claim 1, wherein the large steric hindrance group comprises at least one group selected from the group consisting of biphenyl, triphenyl, o-terphenyl, m-terphenyl, p-terphenyl, spirofluorene, fused dibenzofuran, fused dibenzothiophene, spiroxanthene, adamantane, and soccer olefin.

7. The organic light-emitting diode according to claim 6, wherein R1 and R2 are the large steric hindrance groups.

8. The organic light-emitting diode according to claim 7, wherein m is 1 and the sum of q and p is 2.

9. The organic light-emitting diode according to claim 8, wherein one of R3 and R4 is H and the other is one selected from the group consisting of spirofluorene, fused dibenzofuran, fused dibenzothiophene and spiroxanthene.

10. The organic light-emitting diode according to claim 9, wherein both R1 and R2 are biphenyl.

11. The organic light-emitting diode according to claim 7, wherein L is a single bond, and A1 and A2 are each independently fused aromatic ring groups with C number not less than 18.

12. The organic light-emitting diode of claim 1, wherein in a direction from the anode to the cathode, the organic light-emitting diode comprises:
a hole injection layer, an electron blocking layer, the light-emitting layer, a hole blocking layer, an electron transport layer and an electron injection layer,
wherein the light-emitting layer comprises the blue light host material and a doped material, and the doping ratio of the doped material is 1% to 5%.

13. A method for preparing an organic light-emitting diode according to claim 1, comprising:
forming an anode on a substrate;
forming an electron blocking layer on a side of the anode away from the substrate, and forming a light-emitting layer on a side of the electron blocking layer away from the anode; and
forming a cathode on a side of the light-emitting layer away from the anode.

14. A display panel, comprising:
a substrate having a plurality of organic light-emitting diodes on the substrate, and a part of the plurality of organic light-emitting diodes being those according to claim 1.

15. A display device, comprising a display panel according to claim 14.

16. A method for preparing an organic light-emitting diode according to claim 2, comprising:
forming an anode on a substrate;
forming an electron blocking layer on a side of the anode away from the substrate, and forming a light-emitting layer on a side of the electron blocking layer away from the anode; and
forming a cathode on a side of the light-emitting layer away from the anode.

17. A method for preparing an organic light-emitting diode according to claim 3, comprising:
forming an anode on a substrate;
forming an electron blocking layer on a side of the anode away from the substrate, and forming a light-emitting layer on a side of the electron blocking layer away from the anode; and
forming a cathode on a side of the light-emitting layer away from the anode.

18. A method for preparing an organic light-emitting diode according to claim 4, comprising:
forming an anode on a substrate;
forming an electron blocking layer on a side of the anode away from the substrate, and forming a light-emitting layer on a side of the electron blocking layer away from the anode; and
forming a cathode on a side of the light-emitting layer away from the anode.

19. A display panel, comprising:
a substrate having a plurality of organic light-emitting diodes on the substrate, and a part of the plurality of organic light-emitting diodes being those according to claim 2.

20. A display panel, comprising:
a substrate having a plurality of organic light-emitting diodes on the substrate, and a part of the plurality of organic light-emitting diodes being those according to claim 3.
